(19) United States
(12) Patent Application Publication
(10) Pub. No.: US 2005/0163867 A1
(43) Pub. Date: Jul. 28, 2005

(54) Composition and method of treating gastric ulcers in mammals

(76) Inventor: James Schachtel, Aquebogue, NY (US)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 11/041,656

(22) Filed: Jan. 24, 2005

Related U.S. Application Data
(60) Provisional application No. 60/538,562, filed on Jan. 23, 2004.

(51) Int. Cl. A61K 33/10; A61K 33/08; A61K 31/737

(52) U.S. Cl. 424/690; 514/54; 424/693

(57) ABSTRACT

COMPOSITION AND METHOD OF TREATING GASTRIC ULCERS IN MAMMALS Pharmaceutical compositions suitable for use as an antacid and methods of preventing and treating gastric ulceration in mammals are provided. The composition comprises an Al salt as a first therapeutically active component; a Mg-containing material as a second therapeutically active component; and a polysulfonated or polyphosphorylated carbohydrate-containing composition, as a third therapeutically active component.

### CROSS-REFERENCE TO RELATED APPLICATION

This application claims priority of U.S. Provisional Application No. 60/538,562, which was filed Jan. 23, 2004.

### FIELD OF THE INVENTION

This invention relates to pharmaceutical compositions for treating stomach disorders. More particularly, the invention relates to pharmaceutical compositions suitable as an antacid and suitable for gastrointestinal ulcer treatment and prevention in mammals, such as equine species.

### BACKGROUND

Gastric ulceration in equine species is a well-documented clinical syndrome that affects millions of mammals. More specifically, gastric ulceration affects equine species undergoing rigorous physical exertion such as heavy training. Throughout this Application, the Applicant will refer to “equine” and “horses” interchangeably.

Gastric ulceration results from lowering of the gastric mucosal resistance and in equines from excessive exercise and anorexia. In horses the disease complex that includes ulceration of the esophageal, gastric, or duodenal mucosa is referred to as equine gastric ulcer syndrome (EGUS). This syndrome includes both asymptomatic and symptomatic cases; focal or multifocal lesions of squamous mucosa, glandular mucosa, or both, and gastritis. Gastric lesions are common in adult horses, yearlings, and foals of all breeds.

Horses have evolved as continuous secretors of gastric acid to cater to their constant feeding activity. This is substantially different from humans, who are episodic feeders and thus episodic secretors of acid. Thus a horse stomach produces gastric acid not only in response to food stimuli and gastric distention, but also on a continuous basis. When horses are denied access to food for extended periods, gastric pH may fall rapidly and within 48 hours EGUS may develop.

Episodic feeding followed by withdrawal of feed, typical of horses during racing and training, may increase the risk of ulcer development. Racehorses often undergo fasting for several hours before exercise, allowing levels of gastric acid in the stomach to rise.

In addition, intra-abdominal pressure has been found to increase during exercise and actually compresses the stomach. Considering the horse anatomy, this means that the hydrochloric acid and bile acid in the glandular region of the stomach are forced into the squamous or nonglandular portion of the stomach when a horse is exercised or trained. This ultimately causes gastritis and ulcers to form in the nonglandular portion of the stomach. In addition, this provides strong evidence of why the ulcers are found on the nonglandular portion of the stomach in frequently trained horses, while those turned out to pasture do not have them.

EGUS spans a spectrum of severity, ranging from inflamed but intact epithelium to single, superficial erosions of the mucosal surface, and from there to multiple actively hemorrhaging hyperemic and/or necrotic craters extending beneath the mucosal surface. Perforation may occur, and when it does it is usually fatal.

Chronic gastritis is a frequent concomitant of a gastric ulcer, and is associated with impaired mucous secretion, excessive exercise, and refluxed bile from the small intestine. In experimental animals, it has been demonstrated that protein depletion, avitaminoses, general malnutrition, and heavy exercise increase the susceptibility to gastric ulceration.

Persons skilled in the art are aware of acid-binding active constituents for antacid preparations. Particularly suitable as acid-binding active constituents are magnesium hydroxide, magnesium oxide, magnesium carbonate, magnesium silicate, aluminum hydroxide, aluminum phosphate and magnesium aluminum silicate or mixtures thereof. Preferred active constituents are aluminum hydroxide, magnesium hydroxide, hydrotalcite or magaldrate, which is a magnesium-aluminum silicate.

Oral administration or consumption of acid neutralizing agents (antacids) to treat excess gastric acid and relieve its associated discomfort is well known. Generally, antacid compositions include, as active ingredients, one or more alkaline substances in combination with other inactive ingredients. Alkaline components of the antacid composition effect gastric acid neutralization while the inactive ingredients serve either as a carrier to facilitate administration or to enhance the composition's appeal, palatability, dispensability, and ease of manufacture.

Ideally, an antacid provides rapid and long-lasting relief from the discomfort associated with excess stomach acid. In addition, an effective antacid provides rapid and long-lasting relief in a convenient administrable form and dosage.

A variety of alkaline substances have been previously employed as active ingredients in antacid formulations. For example, U.S. Pat. No. 4,801,608 to Bos et al., the entire disclosure of which is incorporated herein by reference, describes a bismuth containing composition for treating peptic ulcers. Aluminum hydroxide containing antacid compositions are described in U.S. Pat. Nos. 4,514,389 and 4,576,819, the entire disclosures of which are incorporated herein by reference.

Generally, antacid compositions containing weak acid neutralizing agents such as calcium carbonate and aluminum hydroxide are slow acting and, consequently, do not provide rapid relief to the discomfort associated with excess stomach acid. More rapid acting antacids may include magnesium hydroxide, a stronger acid-neutralizing agent. Although primarily incorporated into calcium carbonate containing antacids for its anti-constipative effect, magnesium hydroxide is also known for its antacid activity.

Sucralfate is a basic aluminum sucrose sulfate. Sucralfate has a demonstrated activity as a peptic ulcer prevention and treatment agent having a stromal protein protecting effect (gastric mucosa protecting effect), roles in absorption of bile acids, maintenance of mucosal vascular integrity and of blood flow, which ensures rapid epithelial restitution to repair superficial defects, increase of bicarbonate and mucus secretion, and enhancement of binding of fibroblast growth factor and epidermal growth factor which stimulates angiogenesis, granulation tissue, and epithelization for ulcer healing, a gastric juice pepsin activity inhibiting effect, and possibly an antacid effect.

As a result, sucralfate can be used for alleviation of gastric ulcer, duodenal ulcer, or gastric mucosal lesions in diseases, such as erosion, bleeding, redness or edema; for treatment of acute gastritis; and at the acute aggravated stage of chronic gastritis. Sucralfate shows a therapeutic effect by forming a bioadhesive paste upon reaction with gastric acid in the digestive tract to generate a local protective barrier, which protects the mucous membrane of the digestive tract from an excess of gastric acid, and promotes the in vivo repairing action on the mucosa.

### SUMMARY OF THE INVENTION

This invention provides for a pharmaceutical composition for treating or preventing gastric ulcers in mammals, such as the equine species. More specifically, the pharmaceutical composition provides for a physiologically acceptable excipient two stage pharmacological prevention and treatment action, wherein the composition substantially and immediately decreases acid and aids to coat the stomach while the nonglandular or squamous portion of the stomach is exposed to acid, for example during exercise. The composition can decrease the aggressive forces acting on the stomach and enhance the innate protective forces of mammals taking the composition. The invention therefore provides for a unique and effective treatment and prevention measure for mammalian, especially equine, gastric ulcers.

### DETAILED DESCRIPTION

It will be appreciated that the following description is intended to refer to specific embodiments of the invention selected for illustration in the drawings and is not intended to define or limit the invention, other than in the appended claims.

One particular aspect of this invention relates to a novel antacid composition comprising, in association with a physiologically acceptable excipient, a therapeutically effective amount of a mixture of (a) an alkaline Al salt as a first therapeutically active component; (b) a Mg-containing material as a second therapeutically active component; and (c) a polysulfonated or polyphosphorylated carbohydrate-containing composition, of which sucralfate is an exemplary compound, as a third therapeutically active component.

Al salts suitable for use in the invention include aluminum hydroxide, aluminum phosphate or aluminum carbonate salts. Mg-containing materials suitable for use in the invention include MgO and Mg(OH)₂, which are known to be effective as antacid products.

The carbohydrate polysulfonate compound, such as sucralfate, is not restricted to one particular form, and the use of any sucralfate form is contemplated. Examples are dry sucralfate powder obtained by spray drying undried sucralfate powder, which has been obtained by reacting basic aluminum chloride with sucrose polysulfuric ester, in accordance with the method described in Japanese Patent Publication No. 44-11673 or Japanese Patent Publication No. 44-16037, the entire disclosures of which are incorporated herein by reference; sucralfate designated by the Japanese Pharmacopoeia; fine sucralfate powder formed by further pulverizing dry sucralfate powder, which has been obtained by a conventional method, in accordance with the method described in Japanese Unexamined Patent Publication No. 8-104637, the entire disclosure of which is incorporated herein by reference; and a preparation prepared by adding a plasticizer to the fine powder, mixing them, and then granulating the mixture in molten (or adherent) condition under heat.

The ratio by weight of Al salt to Mg-containing material can be between about 0.8:1.3 and about 1.3:0.8. Preferably, the ratio is between about 0.9:1.1 and about 1.1:0.9, and is most preferably about 1:1. The ratio of the Al salt to the Mg containing material can vary depending on treatment requirements.

The ratio by weight of the first or second therapeutically active component to the third therapeutically active component can be between about 1:0.5 and about 1:1.5. Preferably, the ratio by weight of the first or second therapeutically active component to the third therapeutically active component is between about 1:0.5 and about 1:0.8, and most preferably is between about 1:0.6 and about 1:0.75.

The alkaline Al salt may be selected from the following non-limiting examples: aluminum carbonate, aluminum hydroxide, aluminum phosphate, aluminum hydroxy carbonate, aluminum citrate, dihydroxyaluminum sodium carbonate, aluminum magnesium glycinate, dihydroxyaluminum aminoacetate, dihydroxyaluminum aminoacetic acid, bismuth aluminate, and mixtures thereof. The Mg-containing compound may be seelcted from the following non-limiting examples: hydrated magnesium aluminate, activated sulfate, magnesium aluminate, magnesium aluminosilicates, magnesium carbonate, magnesium glycinate, magnesium hydroxide, magnesium oxide, magnesium trisilicate, and mixtures thereof. The compositions of the invention can also comprise alkaline buffering salts, such as bismuth carbonate, bismuth subcarbonate, bismuth subgallate, bismuth subnitrate, calcium carbonate, calcium hydroxide, calcium phosphate, calcium citrate, calcium citrate malate, potassium carbonate, potassium phosphate, potassium citrate, sodium carbonate, sodium bicarbonate, sodium phosphate, sodium citrate and mixtures thereof.

The compositions of the invention are formulated for administration by any enteral route, including without limitation, oral, buccal, sublingual, and rectal. The physicochemical properties of nutritional compositions, their formulations, and the routes of administration are important in absorption. Absorption refers to the process of nutritional composition movement from the site of administration toward the systemic circulation. Most orally administered nutritional compositions are in the form of tablets, capsules, or an edible paste, primarily for convenience, economy, stability, and patient acceptance. In one preferred aspect, the composition is contained within an edible viscous liquid suitable for consumption by an equine species. Use of the compositions with any of the above routes of administration or dosage forms is performed using well-known procedures and techniques available to the ordinary skilled artisan.

The compositions can comprise any pharmaceutically acceptable carriers, which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders well known to persons skilled in the art, and combinations thereof. Exemplary non-limiting solvents are water, glycerin, ethanol, isopropyl alcohol, methylene chloride or mixtures and combinations thereof. Exemplary non-limiting bulking substances include sugar, syrup, lactose, gelatin, starch, silicon dioxide and combinations thereof.

Plasticizers suitable for use in the invention can be previously dissolved in an organic solvent and added in solution form. Preferred plasticizers may be selected from diethyl phthalate, diethyl sebacate, triethyl citrate, cronotic acid, propylene glycol, butyl phthalate, dibutyl sebacate, caster oil and mixtures thereof, without limitation. As is evident, the plasticizers may be hydrophobic as well as hydrophilic in nature. Water-insoluble hydrophobic substances, such as diethyl phthalate, diethyl sebacate and caster oil are used to delay the release of water-soluble vitamins, such as vitamin B and vitamin C. In contrast, hydrophilic plasticizers are used when water-insoluble vitamins are employed which aid in dissolving the encapsulated film, making channels in the surface, which aid in nutritional composition release.

In a preferred embodiment, the present composition can include aluminum hydroxide (Al(OH)₃) as a first therapeutic ingredient, magnesium hydroxide (Mg(OH)₂) as a second therapeutic ingredient, and a stomach protectant, sucralfate, as a third therapeutic ingredient that cumulatively work quickly to reduce acid and place a protective coating over any existing ulcers and enhance the protective capacities of the stomach.

Al(OH)₃ and Mg(OH)₂ are examples of basic salts that have the ability to neutralize acid and have been shown to decrease the amount of acid in the mammal stomach. One skilled in the art will recognize a number of other basic salts suitable for use as the first or second therapeutic ingredient, some of which are listed above, which have been recognized as suitable to neutralize acid in a mammals stomach. Sucralfate adheres to ulcerated areas of the stomach.

It is noted that the Mg(OH)₂ antacid is generally not absorbed in the intestines and, thus, acts as an osmotic laxative. Additionally, it has been found to increase the motility of the gastrointestinal tract (GIT). On the contrary, Al(OH)₃ has been found to have the opposite effect. It readily crosses the intestinal lining, thereby pulling water from the intestines and it also has been found to decrease gut motility. Thus, one aspect of this invention provides for a substantially equal mixture of these two antacids, which acts to decrease the amount of acid in the gastrointestinal system and allow the treated mammal to maintain an active physiological state during treatment. The other main ingredient is a compound known as sucralfate. This drug has a myriad of important anti ulcer effects, which have been described in the art. As an example, it has been demonstrated that sucralfate adheres to and, thus, protects ulcerated areas of the GIT, helps form a protective coating on normal GIT lining, and exhibits the other beneficial properties described above. It has also been found to absorb bile acids and decrease pepsin production, which have both been found to damage the lining of the stomach, as well as other protective properties.

The Applicant has discovered that the aforementioned combination of a first therapeutic ingredient, such as Al(OH)₃, a second therapeutic ingredient, such as Mg(OH)₂, and a third therapeutic ingredient, such as sucralfate, can be combined into an effective compound to stimulate the innate physiological antacid functions of a mammal, such as an equine species. As such, the composition is designed for administration to an equine species when the equine species exhibits signs of gastric ulcers or is at risk for developing gastric ulcers. The ordinarily skilled veterinarian can readily evaluate the symptoms of gastric ulcers in a subject or determine when a subject is at risk for developing gastric ulcers.

When the equine species is treated with the aforementioned combination of elements, the appetite is stimulated. Consequently, when the equine species, particularly a horse, masticates food, one of the most potent bodily made antacids, saliva, is produced with it. This is swallowed and begins to lower the amount of acid in the stomach. In addition, food in the stomach has also been shown to decrease bile salts and their corrosive effects on the stomach. Thus, the innate gastric protective forces in a subject can be enhanced by administration of the present compositions.

As noted above, intra-abdominal pressure can increase during exercise and compress the stomach, forcing hydrochloric acid and bile acid from the glandular region of the stomach into the squamous or nonglandular portion of the stomach. The mixture can be administered strategically prior to exercise to prevent or treat gastritis and ulcers in the nonglandular portion of the stomach. Provision of the mixture prior to exercise can buffer the hydrochloric and bile acids and decrease the aggressive affects of the stomach contents while the nonglandular region is vulnerable.

The effective amount of the composition can depend on absorption, inactivation and excretion rates of the therapeutically active components, as well as other factors known to those of skill in the art. The effective amount can also vary with the severity of existing ulcers or acidic condition to be alleviated. It is also understood that for any particular subject, specific dosage regimens can be adjusted over time according to individual need, and that the concentration ranges set forth herein are exemplary only and are not intended to limit the scope or practice of the claimed methods. The effective amount of the composition can be administered in a single dose, or can be divided into a number of smaller doses to be administered at varying time intervals. As sometimes used herein, an “effective amount” or “therapeutically effective amount” is that amount of the present compositions sufficient to relieve or eliminate the symptoms of gastric ulceration in mammals, in particular equines, or to prevent or delay the onset or formation of gastric ulcers in mammals, particularly equines.

Thus, specific dosage of the first and second therapeutic agents, such as Mg(OH)₂ and Al(OH)₃, given to horses can vary. As an example, a horse can be given anywhere from about 40 mg to about 30 g (grams), 2-6 times a day, of each of Mg(OH)₂ and Al(OH)₃. The third therapeutic ingredient, such as sucralfate, may be given at a dose ranging from about 2 to about 222 g, 2-6 times a day.

The composition is suitable for administration in post colic surgery cases, in horses who are on a vigorous showing or training schedule, and those horses recovering from “choke,” colitis, enteritis, and any condition which may cause a horse to be off-feed for an extended period of time. Non-limiting examples of specific dosages of the composition are as follows. For treatment of ulcers, about 60 ml can be administered to an approximately 500 kg horse shortly before exercise, such as 15-30 minutes before exercise; and additionally, about 30 ml can be administered at an extended period of 2-12 hours, preferably 6-8 hours, before or after exercise. For horses that are not eating for any reason or an unknown reason, about 30 ml can be administered to an approximately 500 kg horse two to six times per day, more preferably three times per day. For prevention of ulcers, about 45 ml can be administered to an approximately 500 kg horse once daily shortly before exercise.

Generally, the amount of the present compositions to be administered may be adjusted upward or downward depending on the weight of the horse to be treated. For example, for the treatment of ulcers, about 0.1 to about 0.2 ml of the compositions per kg may be administered to a horse shortly before exercise as indicated above. Additionally, about 0.02 to about 0.1 ml of the composition per kg may be administered to a horse over an extended period of time as indicated above. About 0.02 to about 0.1 ml of the composition per kg may be administered to a horse that is not eating, two to six times per day, more preferably three times per day. About 0.05 to about 0.13 ml of the composition per kg may be administered to a horse once daily before exercise, for prevention of ulcers. It is acceptable to administer a dose of the present compositions based on an approximation of the weight of the horse, as precise dosing is not required. Higher or lower doses may be administered, with different dosage regimens, depending on the needs of the horse, the training and feeding schedule, and other factors recognized by one skilled in the art as affecting stomach acid production or the onset or formation of gastric ulcers.

For purposes of illustration the Applicant has provided the following examples of the pharmaceutical composition of this invention. It should be understood that the examples set forth herein are non-limiting examples, and in no way limit the scope of this invention. The following descriptions can be utilized as illustration and guide work for creating an acceptable pharmaceutical composition for the prevention and treatment of ulcers in mammals.

### EXAMPLE 1

A composition was prepared as a suspension of about 1 part Mg(OH)₂ and about 1 part Al(OH)₃ combined with about 1.25 parts of sucralfate, along with various inactive ingredients. One gallon of the composition comprises:
MAGNESIUM HYDROXIDE (Mg(OH)₂)—512 g
ALUMINUM HYDROXIDE (Al(OH)₃)—512 g
SUCRALFATE—640 g
GLYCERIN—1000 ml
APPLE FLAVOR—115 ml
SIMPLE SYRUP at a SUFFICIENT QUANTITY TO MAKE 3840 ml (1 GALLON).

The composition described above was administered to horses known to be suffering from gastric ulceration to alleviate the clinical signs associated with gastric ulcers. Horses weighing approximately 500 kg were given 30 ml of the composition 2-6 times a day. Upon administering the composition to the horse, the horse exhibited increased activity levels, restored balance between peptic aggression and epithelial defense, and the eventual elimination of such symptoms as colic, poor appetite, poor bodily condition, intermittent diarrhea, dullness/attitude change, and poor racing performance.

The Applicant has discovered that the aforementioned composition is readily consumed by an equine species suffering from gastric ulcers, and in turn the symptoms caused by the gastric ulcer are alleviated, and the equine's appetite and activity levels are improved.

### EXAMPLE 2

A composition was prepared as a suspension of about 1 part Mg(OH)₂ and about 1 part Al(OH)₃ combined with about ⅔ part sucralfate, along with various inactive ingredients. As an example, one gallon comprises:
MAGNESIUM HYDROXIDE (Mg(OH)₂)—640 g
ALUMINUM HYDROXIDE (Al(OH)₃)—640 g
SUCRALFATE—426 g
GLYCERIN—1000 ml
APPLE FLAVOR—115 ml
SIMPLE SYRUP at a SUFFICIENT QUANTITY TO MAKE 3840 ML (1 GALLON).

The composition described above was administered to horses known to be suffering from gastric ulceration to alleviate the clinical signs associated with gastric ulcers. Horses weighing approximately 500 kg were given 30 ml 2-6 times a day. Upon administering the composition to the horses, increased activity levels, restored balance between peptic aggression and epithelial defense, and the eventual elimination of such symptoms as colic, poor appetite, poor bodily condition, intermittent diarrhea, dullness/attitude change, and poor racing performance were noted.

The composition is readily consumed by an equine species suffering from gastric ulcers. Administration of the composition relieved the symptoms caused by the gastric ulcer and returned to normal the equine's appetite and activity levels even more effectively than the composition of Example 1.

A variety of modifications to the embodiments described will be apparent to those skilled in the art from the disclosure provided herein. Thus, the invention may be embodied in other specific forms without departing from the spirit or essential attributes thereof and, accordingly, reference should be made to the appended claims, rather than to the foregoing specification, as indicating the scope of the invention.

1. A pharmaceutical composition suitable for use as an antacid comprising:
(a) a first therapeutically active component comprising an alkaline Al salt;
(b) a second therapeutically active component comprising a Mg-containing material, the ratio by weight of the first therapeutically active component to the second therapeutically active component being between about 0.8:1.3 and about 1.3:0.8; and
(c) a third therapeutically active component comprising a polysulfonated or polyphosphorylated carbohydrate-containing composition, the ratio by weight of the first or second therapeutically active component to the third therapeutically active component being between about 1:0.5 and about 1:1.5.

2. The pharmaceutical composition of claim 1 wherein the third therapeutically active component comprises a polysulfonated carbohydrate-containing composition.

3. The pharmaceutical composition of claim 1 wherein the ratio by weight of the first therapeutically active component to the second therapeutically active component is between about 0.9:1.1 and about 1.1:0.9.

4. The pharmaceutical composition of claim 1 wherein the ratio by weight of the first or second therapeutically active component to the third therapeutically active component is between about 1:0.5 and about 1:0.8.

5. The pharmaceutical composition of claim 1 wherein the ratio by weight of the first or second therapeutically active component to the third therapeutically active component is between about 1:0.6 and about 1:0.75.

6. The pharmaceutical composition of claim 1, wherein the first therapeutically active component is Aluminum hydroxide (Al(OH)₃).

7. The pharmaceutical composition of claim 6, wherein the second therapeutically active component is Magnesium hydroxide (Mg(OH)₂).

8. The pharmaceutical composition of claim 7, wherein the third therapeutically active component is sucralfate.

9. The pharmaceutical composition according to claim 8, wherein the ratio between said magnesium hydroxide to said aluminum hydroxide to said sucralfate is about 1.0:1.0:0.67.

10. The pharmaceutical composition according to claim 1 further comprising glycerin.

11. The pharmaceutical composition according to claim 1 further comprising a flavoring additive.

12. The pharmaceutical composition according to claim 1 further comprising syrup.

13. An edible antacid preparation suitable for consumption by an equine species comprising:
about 1 part magnesium hydroxide (Mg(OH)₂);
about 1 part aluminum hydroxide (Al(OH)₃); and
about ⅔ part sucralfate.

14. A method for treating an equine species having gastric ulceration or at risk for developing gastric ulcertation, comprising the step of administering a therapeutically effective amount of the composition of claim 1 to said equine species.

15. A method for stimulating the appetite of an equine species having gastric ulcers or at risk for developing gastric ulcers, comprising administering the composition of claim 1 to said equine species.

16. A method of coating existing ulcers and raising the pH of the stomach before exercise, the method comprising administering the composition of claim 1 to said equine species.

17. The method of claim 14 wherein the composition is administered to said equine species at a dose of about 30 ml, 2-6 times a day.

18. The method of claim 14 wherein the composition is administered to said equine species at a dose of about 45 ml once a day.

19. The method of claim 14 wherein the composition is administered to said equine species at a dose of about 60 ml shortly before exercise and a dose of about 30 ml at an extended period before or after exercise.

20. An antacid composition comprising, in association with a physiologically acceptable excipient, a therapeutically effective amount of a mixture of:
(a) an Al salt as a first therapeutically active component;
(b) a Mg-containing material selected from the group consisting of MgO, Mg(OH)₂, and mixtures thereof as a second therapeutically active components; and
(c) a basic aluminum sucrose sulfate, wherein the weight ratio of the Al salt to the Mg-containing material to the sucralfate is between 1.3:0.8:0.5 to about 0.8:1.3:0.8.

21. A pharmaceutical composition suitable for use as an antacid comprising:
Magnesium hydroxide (Mg(OH)₂);
Aluminum hydroxide (Al(OH)₃);
and sucralfate, wherein the ratio between said magnesium hydroxide to said aluminum hydroxide to said sucralfate is between about 1.1:0.9:0.6 to about 0.9:1.1:0.75.

22. A therapeutic solution for treating an equine species having gastric ulceration or at risk for developing gastric ulcertation, comprising:
magnesium hydroxide (Mg(OH)₂);
aluminum hydroxide (Al(OH)₃);
sucralfate;
glycerin;
apple flavor; and
simple syrup.
